(19) United States
(12) Patent Application Publication
(10) Pub. No.: US 2021/0096131 A1
(43) Pub. Date: Apr. 1, 2021

(54) IN VITRO SCREENING METHOD AND KIT FOR EARLY DIAGNOSIS OF ORAL CAVITY TUMOURS

(71) Applicants: Marina DI DOMENICO, Napoli (NA) (IT); Mariarosaria BOCCELLINO, Napoli (NA) (IT); Alfredo DE ROSA, Castelvolturno (CE) (IT)

(72) Inventors: Marina DI DOMENICO, Napoli (NA) (IT); Mariarosaria BOCCELLINO, Napoli (NA) (IT); Alfredo DE ROSA, Castelvolturno (CE) (IT)

(21) Appl. No.: 17/043,961

(22) PCT Filed: Apr. 1, 2019
(86) PCT No.: PCT/IB2019/052662

§ 371 Date: Sep. 30, 2020

(30) Foreign Application Priority Data
Mar. 30, 2018 (IT) 102018000004137

(51) Int. Cl. G01N 33/574

(52) U.S. Cl. CPC G01N 33/57484; G01N 2333/723; G01N 2333/485

(57) ABSTRACT

The present invention falls within the field of the early diagnosis of tumours of the oral cavity. In particular, the invention relates to a method for the diagnosis and/or for predicting the risk of developing tumours of the oral cavity comprising the detection in cell extracts of certain markers of tumours of the oral cavity using immunological assays, for example ELISA (enzyme-linked immunosorbent assay). The invention also relates to the relative kit for the diagnosis and/or for predicting the risk of developing tumours of the oral cavity.

### FIELD OF THE INVENTION

The present invention falls within the field of the early diagnosis of tumours of the oral cavity. In particular, the invention relates to a method for the diagnosis and/or for predicting the risk of developing tumours of the oral cavity comprising the detection in cell extracts of certain markers of tumours of the oral cavity using immunological assays, for example ELISA (enzyme-linked immunosorbent assay). The invention also relates to the relative kit for the diagnosis and/or for predicting the risk of developing tumours of the oral cavity.

### PRIOR ART

Carcinoma of the oral cavity is among the 10 most common forms of tumour in the world, in fact, over 500,000 new cases are diagnosed each year. Among the tumour manifestations of the head-neck district, various tissues may be affected by malignant tumours of the oral cavity: the oral, lingual, pharyngeal, laryngeal and palatal mucosa and the glandular epithelium. Eighty percent of carcinomas of the oral cavity are squamous cell carcinomas (SSCs), which show a high mortality at 5 years from the prognosis due to the late diagnosis, as there are currently no specific markers. At present there do not exist scientifically reliable, non-invasive screening methods for preventing tumours of the oral cavity.

Thus, there exists the need to provide a method for an early diagnosis of tumours of the oral cavity, which enables subjects at risk of developing the pathology to be identified and subjected to a diagnosis of certainty, based on histological sampling and anatomopathological confirmation.

### DESCRIPTION OF THE INVENTION

The aim of the present invention is to offer an innovative, rapid, economical, sensitive and non-invasive kit, useful for the screening of the early diagnosis to identify neoplastic forms in pre-symptomatic stages. In particular, the authors focused on the expression of specific markers of the carcinoma of the oral cavity, namely the epithelial growth factor receptor (EGFR) and steroid receptors, androgen receptor (AR) and estrogen receptor (ER), proteins that the kit of the invention allows to evaluate, in particular by means of the ELISA assay.

In the context of the present invention, the EGFR sequence preferably corresponds to the sequence available in the NCBI database with Accession No.: NM_005228.3 or the coded protein sequence.

In the context of the present invention, the AR sequence preferably corresponds to the sequence available in the NCBI database with Accession No.: NM_000044.4 or the coded protein sequence.

In the context of the present invention, the ER sequence preferably corresponds to the sequence available in the NCBI database with Accession No.: XP_495993 or NP_001428.1 or the coding nucleotide sequence.

The present invention thus provides a non-invasive diagnostic aid for screening for the possible presence of pre-cancerous lesions, useful in supporting the early diagnosis of the tumours of the oral cavity, with a view to tertiary prevention. The method and the kit of the invention are in fact aimed in particular at subjects who have inflammatory conditions that may prelude to cellular transformations.

The present invention falls within the field of devices based on immunological assays, preferably it is based on a protein analysis on cell extracts via ELISA.

The invention further relates to the realization of an innovative product built through the assembly of semi-processed products that are also commercially available. The product comprises an instrument useful for sampling the potentially dangerous cells, and everything necessary to enable the professional (dentist) to analyse them and verify the presence of biomarkers, interpreting the result of the test itself. The product has an extremely simple method of use.

It is therefore an object of the present invention an in vitro method for predicting the risk of developing and/or for the diagnosis and/or for the prognosis and/or for monitoring the progression of and/or for the screening of a therapeutic treatment of a tumour of the oral cavity in a subject comprising the following steps:

a) detecting and/or quantifying at least the androgen receptor (AR) and the estrogen receptor (ER) in a biological sample obtained from the subject, preferably from the oral cavity of the subject; and

b) comparing with respect to a control sample.

Preferably, the sample obtained from the subject is obtained from the oral cavity of the subject, more preferably from the oral, lingual, pharyngeal, laryngeal and palatal mucosa and the glandular epithelium. Preferably, the sample obtained from the subject is obtained from the tongue.

In a preferred embodiment, in step a) the epithelial growth factor receptor (EGF-R) is further detected and/or quantified.

Preferably, the presence of AR and/or ER and/or EGF-R, and/or a higher amount of AR and/or ER and/or EGF-R with respect to the amount in the control sample indicates that the subject is at risk of developing or is suffering from a tumour of the oral cavity.

Preferably, step a) comprises:
contacting and/or incubating said biological sample with at least an anti-AR antibody and/or an anti-ER antibody and/or an anti-EGF-R antibody under conditions such that the AR and/or the ER and/or the EGF-R bind to said antibodies and form an antibody-antigen complex if AR and/or ER and/or EGF-R are present; and
detecting and/or quantifying AR and/or ER and/or EGF-R bound to the antibody, preferably using detection and/or quantification means for said antibodies.

In a further preferred embodiment, the subject in whom the presence of AR and/or ER and/or EGF-R and/or of the relative antibodies as defined above has been detected and/or quantified is subsequently subjected to further methods of diagnosis of a tumour of the oral cavity, such as, for example histological sampling and anatomopathological confirmation. Such diagnostic methods presently used are costly and invasive. Therefore, the method according to the invention enables the performance of a first screening of subjects potentially at risk that is effective, fast and non-invasive.

Preferably, said biological sample comes from an inflamed area of the oral cavity. Also preferably, said biological sample is a cellular, preferably subjected to cellular lysis, or tissue sample or a fluid, for example saliva, blood or serum.

Preferably, said tumour of the oral cavity is selected from the group consisting of: tumour of the oral mucosa, lingual tumour, pharyngeal tumour, laryngeal tumour, palatal tumour, tumour of the glandular epithelium, squamous cell carcinoma (SSC), epidermal carcinoma of the mouth, laryngeal carcinoma, carcinoma of the tongue and carcinoma of the lip.

It is a further object of the present invention a kit comprising:
detection and/or quantification means of AR and ER and optionally EGF-R;
optionally control means.

In a preferred embodiment, the kit comprises:
(a) at least an anti-AR antibody and an anti-ER antibody;
(b) detection and/or quantification means of at least an AR-antibody complex and an ER-antibody complex.

The kit preferably optionally comprises control means.

Preferably, the kit further comprises an anti-EGF-R antibody and detection and/or quantification means of an EGF-R-antibody complex.

Preferably, in the kit according to the invention, the anti-AR antibody and/or the anti-ER antibody and/or the anti-EGF-R antibody are immobilized to a solid support.

Preferably, said solid support is a plastic strip, preferably PVDF (polyvinylidene fluoride). In a particularly preferred embodiment, the kit comprises a device with two ends, wherein the first end comprises the anti-AR antibody and/or the anti-ER antibody and/or the anti-EGF-R antibody and the second end comprises a brush for drawing a biological sample from a subject. Preferably, the first end further comprises a positive control and/or a negative control.

In a further preferred embodiment, the kit comprises: at least a buffer solution and/or a lysis solution and/or a detection system. Preferably, said detection system comprises or consists of a secondary antibody provided with an enzymatic detector system and/or a substrate for the detection of the secondary antibody, for example by colorimetric reaction. Preferably, said buffer solution and/or lysis solution and/or detection system are provided in different wells, preferably placed in a single box.

The present invention further provides a use of the kit as described above to perform the method as described above.

In a preferred embodiment, said use comprises the following steps:
immersion of said biological sample in a lysis solution to obtain a first solution in which AR and/or ER and/or EGF-R are released, if present in the sample;
immersion of said anti-AR and/or anti-ER antibodies and optionally anti-EGF-R in said first solution comprising to obtain first antibody-antigen complexes;
immersion of said first antibody-antigen complexes in a second solution comprising a secondary antibody provided with an enzymatic detector system to obtain second antibody-antigen complexes;
immersion of said second antibody-antigen complexes in a third solution comprising a substrate for the detection of the secondary antibody;
detecting and/or quantifying AR and/or ER and/or EGF-R with respect to a control sample.

A further object of the present invention is a kit as described above for use in the method as described above.

It is a further object of the present invention a device comprising two ends, wherein the first end comprises the anti-AR antibody and/or the anti-ER antibody and optionally the anti-EGF-R antibody and the second end comprises a brush for drawing a biological sample from a subject. Preferably, the first end further comprises a positive control and/or a negative control. Preferably, the first end preferably comprises a PVDF strip loaded with the antibodies defined above.

The present invention further provides a use of the device as described above to perform the method as described above.

In the context of the present invention, the markers “AR”, “EF”, “EGF-R” include the respective gene, mRNA, cDNA or the protein coded by them, including fragments, derivatives, variants, isoforms etc. Preferably, said markers are characterised by the NCBI Accession numbers defined above.

In the present invention, the expression “proteins” preferably refers to the androgen receptor, the estrogen receptor and/or the epithelial growth factor receptor, preferably human, and the expression “antibodies” preferably refers to the relative anti-AR (for example Ab N-20 sc 816; Santa Cruz Biotechnologies Inc.), anti-ER (for example anti-ERα antibody sc 543; Santa Cruz Biotechnologies Inc.) and/or anti-EGFR (for example antibody sc-03-G, Santa Cruz Biotechnologies Inc.) antibodies.

The expression “protein” is understood as also comprising the corresponding protein coded by corresponding orthologous or homologous genes, functional mutants, functional derivatives, functional or analogue fragments, isoforms thereof.

In the context of the present invention, the term “polypeptide” or “protein” comprises:

i. the entire protein, allelic variants and orthologues thereof;

ii. any synthetic, recombinant or proteolytic functional fragment;

iii. any functional equivalent, such as, for example, synthetic or recombinant functional analogues.

In the present invention, the expressions “method for predicting the risk of developing”, “method for the diagnosis”, “method of screening” and/or “screening” preferably comprise the screening of subjects potentially at risk of being affected by or developing a tumour of the oral cavity.

In the present invention, the “control sample” and/or the “control means” can be a sample isolated from a healthy subject or from a patient affected by another disorder or from a patient affected by a tumour of the oral cavity prior to a therapeutic treatment, a patient affected by a tumour of the oral cavity during a therapeutic treatment, a patient affected by a tumour of the oral cavity at different times during the course of the disease. The control means can be used to compare the presence of the markers as defined above with respect to an appropriate control. The control can be obtained for example, with reference to known standards, from both a normal subject and a normal population.

In particular, in the present invention, the expression “positive control” preferably refers to a sample which contains the proteins defined above and/or nucleic acids coding for said proteins and/or to messenger RNAs transcribed by said nucleic acids or to an anti-actin antibody, whereas the expression “negative control” refers to a sample that does not contain them, such as, for example, cells derived from different tumours.

In the case of a method for monitoring the progression of a tumour of the oral cavity, the control sample could be a sample isolated from the same subject at various points in time before the start of the therapy, at various points in time during the course of the therapy, etc.

In the case of a method of screening for a therapeutic treatment of a tumour of the oral cavity, the control sample can be a sample drawn from a subject with no treatment or from a subject treated with a substance to be assayed or from a subject treated with a reference treatment. In this case, if the amount of the markers defined above in the isolated biological sample is lower than or equal to the control value, the tested substance could be effective for treating the tumour.

In the methods according to the invention, when the amount of the markers defined above in the isolated biological sample is higher than the control value, it may indicate that the subject has an unfavourable prognosis.

As used here, the term “subject” comprises any human or non-human being, for example mammals and non-mammals, such as non-human primates, sheep, dogs, cats, horses, cows, chickens, amphibians, reptiles, etc.

In the context of the present invention, the detection means or detection systems preferably comprise means capable of detecting and/or measuring the amount of the markers defined above. In the case where a protein is detected, the detection means are for example at least one antibody that is specific for the protein, functional analogues or derivatives thereof. In the case an antigen-antibody complex is to be detected, the detection means are for example secondary antibodies conjugated to an enzyme, luminescent substrates, magnetic beads coated with capture antibodies, personalised freeze-dried antibody cocktails and/or columns with dimensional filtration cartridges and/or combined with a specific antibody filter (SAF). Preferably, said secondary antibody is provided with an enzymatic detector system and said detection system further comprises a substrate for the detection of the secondary antibody, for example by colorimetric reaction.

The kits according to the invention can further comprise usual auxiliary components, such as buffers, carriers, dyes, etc. and/or instructions for use. Preferably, said kit further comprises a solid support in which the antibody is immobilized. Preferably, the kit of the invention is preferably a kit for immunological assay, more preferably an ELISA kit.

The kit can further comprise control means for comparing the increase in the amount of the markers with an appropriate control value. The control value can be obtained, for example, with reference to known standards, from both a normal subject and a normal population.

In the present invention, the expression “detect” or “detection” in relation to a protein or a nucleic acid (DNA or RNA) or the respective antibodies, refers for example to any method of observation, assessment or quantification of the signals indicative of the presence of the protein in a sample or the absolute or relative amount of said target protein in a sample, for example by chemiluminescence, fluorimetry, spectrophotometry, etc. The methods can be combined with protein or nucleic acid marking methods to provide a signal, for example: immunohistochemical staining, ELISA, cell suspension, cytology, fluorescence, radioactivity, colorimetry, gravimetry, X-ray diffraction or adsorption, magnetism, enzymatic activity and the like. In the present invention, the detection of the presence of messenger RNA transcribed by the nucleic acid or relative proteins or antibodies as defined above can be performed by means of any technique known to the person skilled in the art, such as, for example, Northern blotting or quantitative or semi-quantitative RT-PCR methods using appropriate oligonucleotide primers.

The detection and/or quantification of the markers defined above can correspond to the measurement of the amount or to the measurement of an alteration in the amount of the marker, more in particular to an increase or a decrease in its amount. A detection of an increase may be correlated with a worsening of the disorder, whereas a decrease may be correlated with an improvement of the disorder or the subject's recovery.

In a preferred embodiment, the method further comprises detecting and/or quantifying at least one further marker and comparing with an appropriate control sample.

In the present invention, the expression “quantify” or “quantification” can be understood as a measurement of the amount or concentration either of said receptors or of the respective antibodies, preferably semi-quantitative or quantitative. The term “amount”, as used in the description refers to but is not limited to the absolute or relative amount of proteins or antibodies, and any other value or parameter associated with the same or which can derive from them. Such values or parameters comprise intensity values of the signal obtained from either physical or chemical properties of the protein or antibody, obtained by direct measurement, for example, intensity values in an immunoassay, mass spectroscopy or nuclear magnetic resonance. Moreover, these values or parameters include the ones obtained by indirect measurement.

The antibodies defined above comprise human and animal monoclonal antibodies or fragments thereof, single-chain antibodies and fragments and miniantibodies thereof, bispecific antibodies, diabodies, triabodies, or dimers, oligomers or multimers thereof. Preferably, the antibody is selected from the group consisting of an intact immunoglobulin (or an antibody), an Fv, an scFv (single-chain Fv fragment), an Fab, an F(ab′)₂, an antibody type domain, an antibody mimetic domain, a single antibody domain, a multimeric antibody, a peptide or a proteolytic fragment containing the epitope binding region. In the present invention, the term “antibody” is used in the broadest sense and comprises various antibodies and antibody mimetic structures, comprising, but not limited to, monoclonal antibodies, polyclonal antibodies, multi specific antibodies (for example bispecific antibodies), human antibodies, humanized antibodies, deimmunized antibodies, chimeric antibodies, nanobodies, antibody derivatives, antibody fragments, anticalins, DARPins, ““affibodies””, ““affilins””, affimers, affitins, ““alphabodies””, avimers, finomers, minibodies and other binding domains, on condition that they show the binding activity desired for the antigen.

The present invention will be described through non-limiting examples, making reference to the following figures:

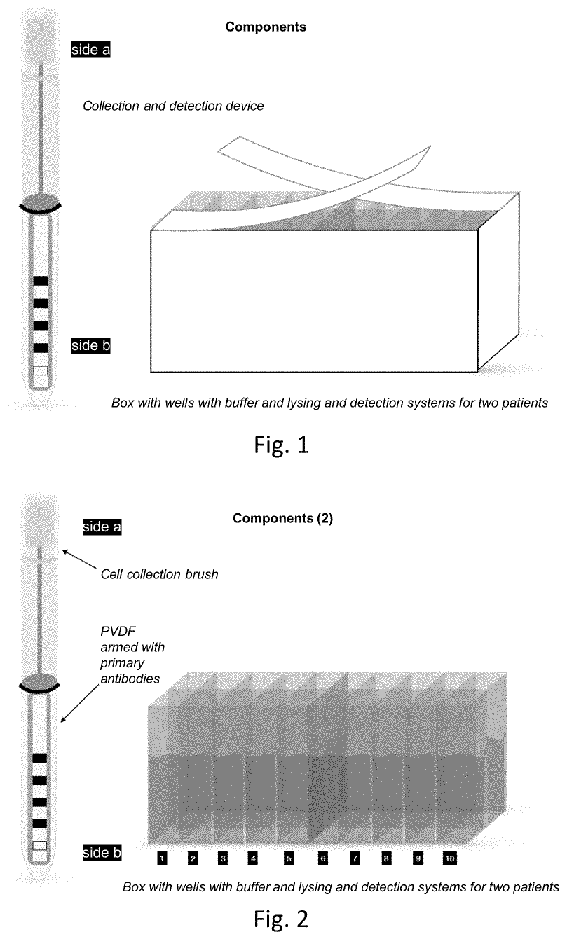

FIG. 1. Components: device provided with a brush (side a) and PVDF strip on plastic support (side b) and detection box.

FIG. 2. Components: device provided with a brush (side a) and PVDF strip on plastic support armed with primary antibodies (side b) and detection box with twenty wells with buffer, lysing and detection systems useful for two patients.

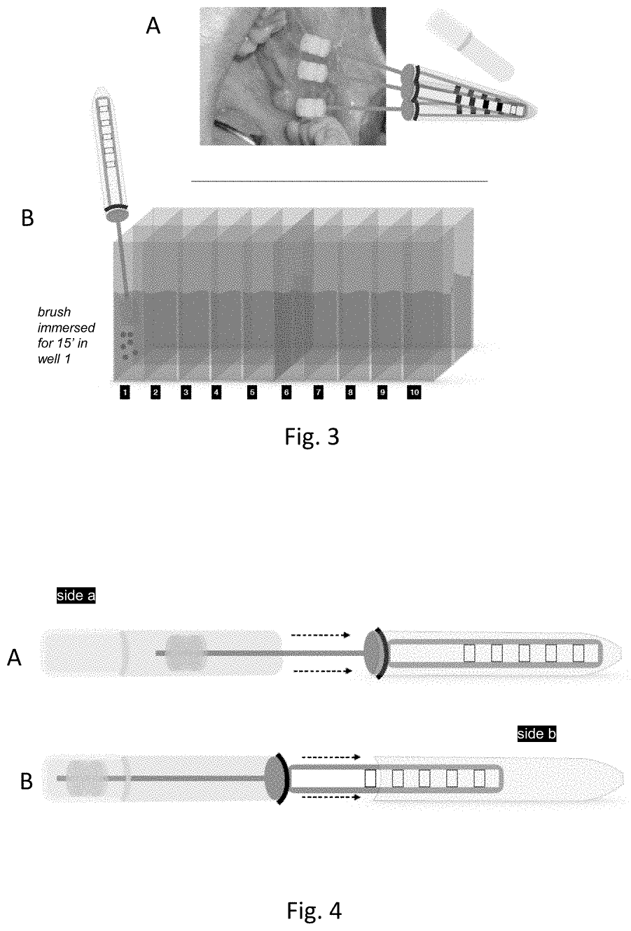

FIG. 3. Operating scheme of the device: (a) drawing of the cells from the oral mucosa with the brush; (b) immersion of the brush in the first well.

FIG. 4. Scheme of use of the device: (a) extraction of the brush; (b) extraction of the PVDF strip.

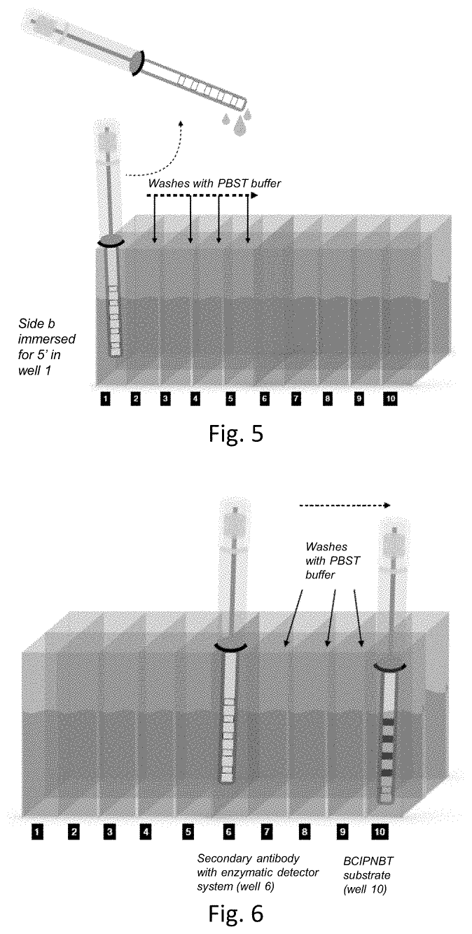

FIG. 5. Scheme of immersion of the PVDF strip in the first well and subsequent four washes.

FIG. 6. Scheme of immersion of the strip in well 6 for the reaction with the secondary antibody, in wells 7, 8 and 9 for the washes and in well 10 for the detection with the substrate.

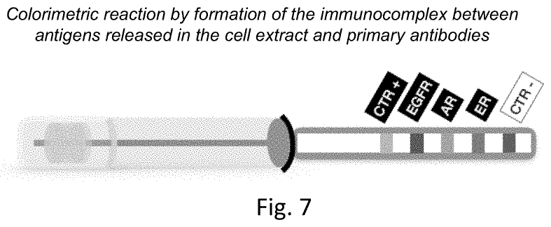

FIG. 7. Colorimetric expression relative to the results.

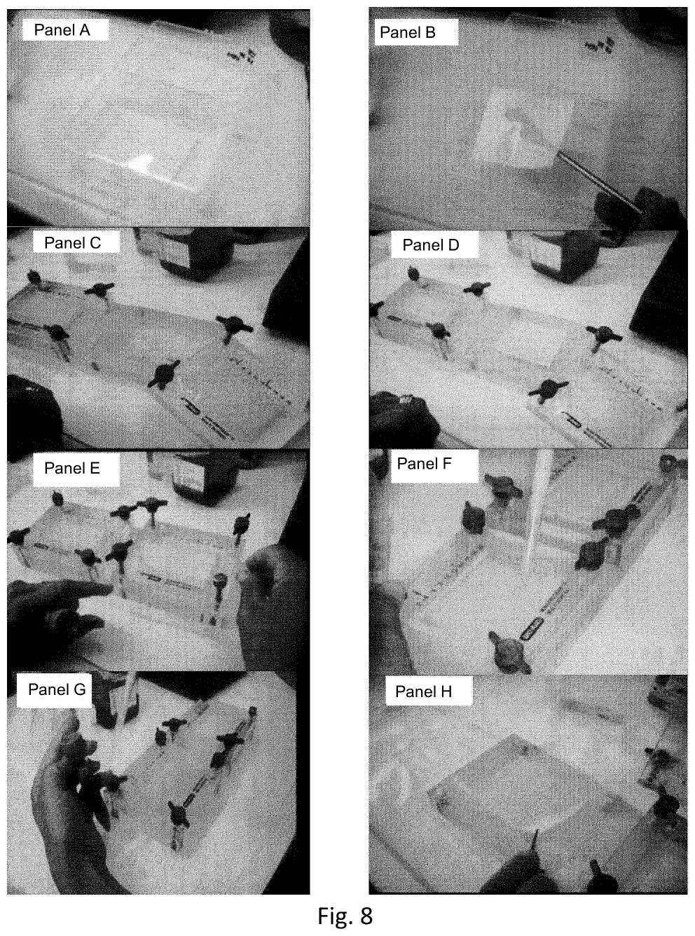

FIG. 8. Steps of preparing the strip.

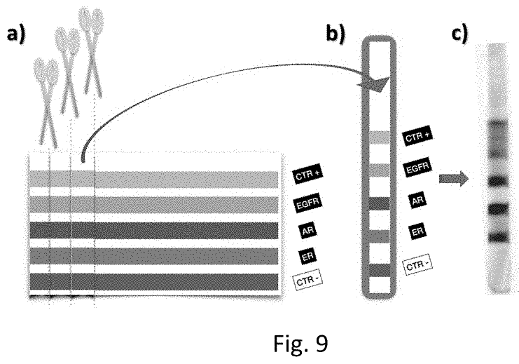

FIG. 9. (a) and (b) Strip formation scheme and (c) subsequent development.

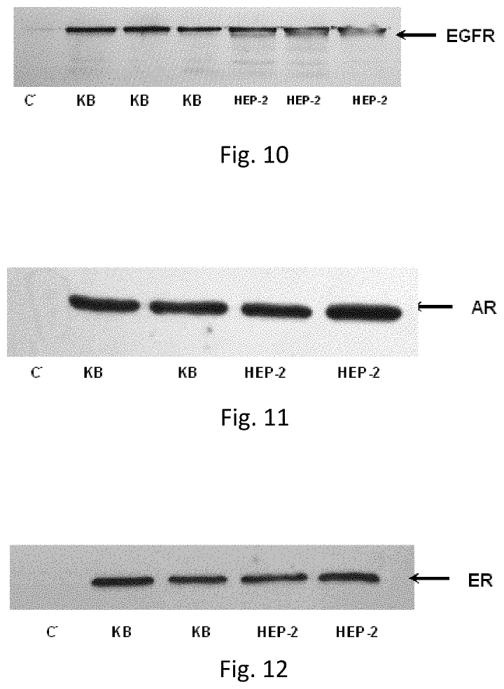

FIG. 10. Western blot for EGFR in KB and HEP-2 cells.

FIG. 11. Western blot for AR in KB and HEP-2 cells.

FIG. 12. Western blot for ER in KB and HEP-2 cells.

### MATERIALS AND METHODS

### Protocol for Preparing the PVDF Strip

The steps for preparing the PVDF strip armed with the primary antibodies of interest are the following:

1) Hydration of the PVDF strip (ThermoFisher Scientific Catalog Number LC2002) with methanol for 5 minutes (FIG. 8, Panel A).

2) Wash with water for 5 minutes (FIG. 8, Panel B).

3) Two washes with PBS for 5 minutes (FIG. 8, Panel B).

4) Incubation with the protein A-biotin from Staphylococcus aureus (Sigma-Aldrich, 10 μg/mL) for 1 hour in PBS (FIG. 8, Panel B).

5) Two washes with PBS (FIG. 8, Panel B).

6) Blocking with 3% BSA solution in PBS for 1 hour (FIG. 8, Panel B).

7) Three washes with PBS for 5 minutes (FIG. 8, Panel B).

8) Mounting of the PVDF on BioRad multi-channel apparatus (FIG. 8, Panels C, D, E).

9) Incubation with solution of rabbit antibodies (anti-AR antibody: Ab N-20 sc 816; Santa Cruz Biotechnologies Inc., Santa Cruz, Calif.; anti-ERα antibody sc 543; Santa Cruz Biotechnologies Inc., Santa Cruz, Calif.; and anti-EGFR antibody sc-03-G, Santa Cruz Biotechnologies Inc., Santa Cruz, Calif.) about 400 μL per channel, under stirring O.N. (FIG. 8, Panel F).

10) Three washes with PBS for 5 minutes in every channel (FIG. 8, Panel F).

11) Two washes of the filter with PBS TEA 0.2 M (FIG. 8, Panel F).

12) Incubation with DMP 25 mM in TEA HCl 0.2 M pH 8.2 (FIG. 8, Panel F, G, H)

13) Incubation with TEA 0.2 M+20 mM ethanolamine (FIG. 8, Panel F, G, H)

14) Two washes with PBS for 5 minutes (FIG. 8, Panel F, G, H).

15) Storage in 0.02 NaN3 in PBS (FIG. 8, Panel F, G, H).

16) The filter is cut (FIG. 9a) perpendicularly to the channels in which the primary anti-EGFR, anti-AR and anti-ER antibodies were loaded (for the capture of the relative antigens released from the collected cytology sample) to obtain a strip about 0.4 cm wide which will be mounted on the support of the device (FIG. 9b). The strip is then treated with ELISA method with the aim of revealing the specific antigens present in the tested cytology sample (FIG. 9c).

The cell lines derived from the mucosa of the oral cavity express the epithelial growth factor receptor (EGFR) and are therefore not suitable as negative control of a screening based on the positivity of the expression of hormone receptors.

The loaded negative control (CTR−) derives from clones of tumour cells deriving from mammary cancer (MDA-MB-453, ATCC HTB-131), whose specific characteristics are listed below:

i) the clone, MDA-MB-453, despite being of epithelial tumour derivation does not express the epithelial growth factor receptor (EGF-R);

ii) the clone, MDA-MB-453, despite being of mammary tumour cell derivation, does not express the androgen receptor (AR) and the estrogen receptor (ER) (Kristina Subik et al., Breast Cancer (Auckl). 2010; 4: 35-41).

The positive control (CTR+) is an anti-actin antibody (Sigma-Aldrich, A2066).

### ELISA Protocol of the PVDF Strip in Cells of the Oral Cavity

KB cells (ATCC CCL-17) derived from the epidermal carcinoma of the mouth and HEP-2 cells (ATCC CCL-23) derived from laryngeal carcinoma, are lysed in a lysis buffer, the extracted proteins are separated by SDS-PAGE. At the end of the electrophoretic run, the proteins are transferred onto a PVDF filter for 2 hours at room temperature. Afterwards, the filter is washed for two hours in a PBS buffer containing Tween-20 pH 7.2 (PBST-buffer, PBS Sigma-Aldrich P5368 and 0.02% Tween-20 Sigma-Aldrich P1379) and 3% bovine serum albumin (BSA) in order to block the nonspecific sites. The filter is subsequently incubated for at least two hours with the specific anti AR, anti ER and anti EGFR primary antibodies (anti-AR antibody: Ab N-20 sc 816; Santa Cruz Biotechnologies Inc., Santa Cruz, Calif.; anti-ERα antibody sc 543; Santa Cruz Biotechnologies Inc., Santa Cruz, Calif.; and anti-EGFR antibody sc-03-G, Santa Cruz Biotechnologies Inc., Santa Cruz, Calif.). Finally, after three 10-minute washes with PBST-buffer, the filter is incubated with the specific secondary antibodies and the signal will be detected by means of a chemiluminescence release kit (ECL, Amersham Pharmacia Inc.). The semiquantitative densitometric analysis was run by means of a Scan LKB (Amersham Pharmacia Inc.).

### DESCRIPTION OF THE DEVICE

### Analysis Instrumentation and Composition Thereof

1) plastic pen-like device with two functional ends one provided with brush and yellow cap (side A) the other provided with support for the PVDF strip (Immobilon) armed with the primary antibodies of interest, with transparent cap (side B) FIG. 1 and FIG. 2;

2) box organized in rows of 10 wells closed off with LED aluminium strips containing buffers and lysing and detection systems; FIG. 1 and FIG. 2.

### Mode of Use

The dentist, in the presence of potentially cancerous lesions, can draw a sample of cells using the specific collection instrument present in the kit and proceed as follows:

1) Collection of the cytology sample: drawing of cells from the oral cavity on the suspect mucosa with the special brush present at the end (side A) of the device provided with a yellow cap; FIG. 3a.

2) Immersion of the brush for 15 minutes in the first well containing the lysis buffer, which enables the release in solution of the proteins, i.e. the antigens of interest, EGFR, AR and ER present in the extract of the cells of the oral cavity; FIG. 3b.

3) The cap of the device side A is closed again; FIG. 4.

4) The cap of the device side B is opened; FIG. 4b.

5) Immersion of the end (side B) of the support with the PVDF strip for 5 minutes in the first well to enable the interaction of the proteins (antigens) with the primary antibodies adhered to the PVDF strip in order to form the immunocomplex (anti-AR antibody: Ab N-20 sc 816; Santa Cruz Biotechnologies Inc., Santa Cruz, Calif.; anti-ERα antibody sc 543; Santa Cruz Biotechnologies Inc., Santa Cruz, Calif.; and anti-EGFR antibody sc-03-G, Santa Cruz Biotechnologies Inc., Santa Cruz, Calif.); FIG. 5.

6) Washes (three) of the support with the PVDF strip in wells containing PBST-buffer to eliminate the proteins non-specifically adhered to the immunocomplex; FIG. 5.

7) Immersion of the support with the PVDF strip for 10 minutes in the well containing the secondary antibody provided with an enzymatic detector system (Alkaline phosphatase, Sigma-Aldrich A2306); FIG. 6.

8) Washes (two) of the support with the PVDF strip in the wells containing PBST-buffer to eliminate the excess secondary antibody; FIG. 6.

9) Immersion of the support with the PVDF strip in the well containing the substrate (BCIP/NBT, ROCHE 11681451001) necessary for the colorimetric reaction; FIG. 6.

10) Washes (two) of the strip in the well containing PBST-buffer to eliminate the excess; FIG. 6.

11) The colorimetric reaction will be noted in the presence of the proteins of interest; FIG. 7.

### RESULTS

The proposed invention is the fruit of scientific studies carried out by the author on the crosstalk between EGF and estradiol (ER) and androgen (AR) receptors in epithelial tumours. Such studies were the translational prerequisite for the formulation of an instrument useful for the early diagnosis of neoplastic pathologies. Such use finds soundness in the results, which demonstrate that the complex of steroid receptors (AR/ER/Src) is required for the action of the EGF (1.2). The use of the ELISA techniques reported in the scientific papers of the author made the realization of the detector system of the device possible.

The cell extracts used in the laboratory selected for the validation of the conceived instrument, come from KB cells derived from epidermal carcinoma of the mouth and from HEP-2, cells derived from laryngeal carcinoma. FIG. 9c shows the results regarding the co-expression of the proteins of interest (EGFR, AR and ER), on the PVDF strip of the device. As confirmation of the results obtained with the device, the cell extracts were subjected to Western blot analysis for the individual receptors and the results are shown in FIGS. 10, 11, 12. In FIG. 10, the bands relative to the EGFR receptor in KB and HEP-2 cells are evident; the samples were loaded in triplicate and the receptor is clearly evident in the two cell types, whereas it does not appear evident in the negative control represented by MDA-MB-453 cells (Kristina Subik et al. Breast Cancer (Auckl). 2010; 4: 35-41). In FIG. 11 one sees the presence in the same cells of the AR receptor (the samples were loaded in duplicate), in this case as well the band is visible only in the cells of the oral cavity (KB and HEP-2) and not in the negative control (MDA-MB-453). Finally, FIG. 12 shows the ER receptor (the samples were loaded in duplicate) in this case as well the receptor was revealed only in the cells of the oral cavity (KB and HEP-2) and not in the negative control (MDA-MB-453).

The test proposed ensures a significantly high index of predictability, since the simultaneous expression of these three receptors correlates with the change in the cell phenotype from benign to malignant.

### REFERENCES

1) Fiorelli A, Ricciardi C, Pannone G, Santoro A, Bufo P, Santini M, Serpico R, Rullo R, Pierantoni G M, Di Domenico M. Interplay between steroid receptors and neoplastic progression in sarcoma tumors. J Cell Physiol. 2011 November; 226(11):2997-3003. doi: 10.1002/jcp.22645.

2) Migliaccio A, Castoria G, Di Domenico M, Ciociola A, Lombardi M, De Falco A, Nanayakkara M, Bottero D, De Stasio R, Varricchio L, Auricchio F. Crosstalk between EGFR and extranuclear steroid receptors. Ann N Y Acad Sci. 2006 November; 1089:194-200.

1. An in vitro method for predicting the risk of developing and/or for the diagnosis and/or for the prognosis and/or for monitoring the progression and/or for the screening of a therapeutic treatment of a tumour of the oral cavity in a subject comprising the following steps:
a) detecting and/or quantifying at least the androgen receptor (AR) and the estrogen receptor (ER) in a biological sample obtained from the subject, optionally from the oral cavity of the subject; and
b) comparing with respect to a control sample.

2. The method according to claim 1, wherein in step a) the epithelial growth factor receptor (EGF-R) is further detected and/or quantified.

3. The method according to claim 1, wherein the presence of AR and/or ER, and optionally EGF-R, and/or a higher amount of AR and/or ER, and optionally EGF-R, with respect to the amount in the control sample indicates that the subject is at risk of developing or is suffering from a tumour of the oral cavity.

4. The method according to claim 1, wherein step a) comprises:
contacting and incubating said biological sample with at least an anti-AR antibody and an anti-ER antibody and optionally an anti-EGF-R antibody under conditions such that the AR and the ER and optionally the EGF-R bind to said antibodies and form an antibody-antigen complex if AR and/or ER and/or EGF-R are present; and
detecting and/or quantifying AR and/or ER and/or EGF-R bound to the antibody, preferably using detection and/or quantification means for said antibodies.

5. The method according to claim 1, wherein the subject in whom the presence of AR and/or ER and optionally EGF-R and/or of an anti-AR antibody and/or an anti-ER antibody and optionally an anti-EGF-R antibody has been detected and/or quantified is subsequently subjected to further methods of diagnosis of a tumour of the oral cavity.

6. The method according to claim 1, wherein said biological sample originates from an inflamed area of the oral cavity.

7. The method according to claim 1, wherein said biological sample is a cellular, optionally subjected to cellular lysis, or a tissue sample or a fluid, said fluid optionally being one of saliva, blood or serum.

8. The method according to claim 1, wherein said tumour of the oral cavity is selected from the group consisting of: tumour of the oral mucosa, lingual tumour, pharyngeal tumour, laryngeal tumour, palatal tumour, tumour of the glandular epithelium, squamous cell carcinoma (SSC), epidermal carcinoma of the mouth, laryngeal carcinoma, carcinoma of the tongue and carcinoma of the lip.

9. A kit comprising:
detection and/or quantification means of AR and ER and optionally EGF-R;
optionally control means.

10. The kit according to claim 9 comprising:
(a) at least an anti-AR antibody and an anti-ER antibody; and
(b) detection and/or quantification means of at least an AR-antibody complex and an ER-antibody complex.

11. The kit according to claim 10 further comprising an anti-EGF-R antibody and detection and/or quantification means of an EGF-R-antibody complex.

12. The kit according to claim 10, wherein the anti-AR antibody, the anti-ER antibody and optionally the anti-EGF-R antibody are immobilized to a solid support.

13. The kit according to claim 9, comprising a device with two ends, wherein the first end comprises the anti-AR antibody, the anti-ER antibody and optionally the anti-EGF-R antibody and the second end comprises a brush for drawing a biological sample from a subject, optionally, the first end further comprises a positive control and/or a negative control.

14. The kit according to claim 9 comprising at least a buffer solution and/or a lysis solution and/or a detection system.

15. The kit according to claim 14 wherein said buffer solution and/or lysis solution and/or detection system are provided in different wells, optionally, placed in a single box.

16. (canceled)

17. (canceled)

18. (canceled)

19. A device comprising two ends, wherein the first end comprises the anti-AR antibody, the anti-ER antibody and optionally the anti-EGF-R antibody and the second end comprises a brush for drawing a biological sample from a subject, wherein the first end optionally further comprises a positive control and/or a negative control.

20. (canceled)

21. An in vitro method for predicting the risk of developing and/or for the diagnosis and/or for the prognosis and/or for monitoring the progression and/or for the screening of a therapeutic treatment of a tumour of the oral cavity in a subject comprising:
a) immersing a biological sample in a lysis solution to obtain a first solution in which the androgen receptor (AR) and/or the estrogen receptor (ER) and/or epithelial growth factor receptor (EGF-R) are released, if present in the biological sample;
b) immersing the anti-AR and anti-ER antibodies and optionally anti-EGF-R in said first solution to obtain first antibody-antigen complexes;
c) immersing said first antibody-antigen complexes in a second solution comprising a secondary antibody provided with an enzymatic detector system to obtain second antibody-antigen complexes;
d) immersing said second antibody-antigen complexes in a third solution comprising a substrate for the detection of the secondary antibody; and
e) detecting and/or quantifying the AR and/or ER and/or EGF-R with respect to a control sample.
